(19) United States
(12) Patent Application Publication
(10) Pub. No.: US 2018/0310808 A1
(43) Pub. Date: Nov. 1, 2018

(54) STERILE ADAPTER

(71) Applicant: Xion GmbH, Berlin (DE)

(72) Inventors: Helmut Laser, Berlin (DE); Holger Muller, Glienicke-Nordbahn (DE)

(21) Appl. No.: 15/771,468

(22) PCT Filed: Oct. 26, 2016
(86) PCT No.: PCT/EP2016/075743

§ 371 Date: Apr. 27, 2018

(30) Foreign Application Priority Data
Nov. 13, 2015 (DE) 10 2015 119 695.6

(51) Int. Cl. A61B 1/00; A61B 1/04

(52) U.S. Cl. CPC A61B 1/00142; A61B 46/10; A61B 1/042

(57) ABSTRACT

A sterile adapter for arrangement between a sterilizable endoscope and a camera head with a distal end attachable to the endoscope, and a proximal end attachable to the camera head, wherein the sterile adapter has a tube with a longitudinal axis, with a longitudinal portion having an internal diameter reduced in a plane perpendicular to the longitudinal axis and which forms a central opening which is dimensioned such that, while a free propagation of light is possible between the endoscope and the camera head along the longitudinal axis, it is nonetheless made difficult to reach through an interior of the sterile adapter enclosed by the tube.

The invention relates to a sterile adapter for arrangement between a sterilizable endoscope and a camera head.

In medical endoscopy, instruments that can be touched by the patient or operator have to be sterile. However, the widely used method of steam sterilization requires considerable effort. An arrangement commonly used in endoscopy involves a combination of an endoscope and of a camera head attachable to the latter. In this arrangement, the endoscope is usually sterilized, but the camera head is covered with a disposable sterile cover. It is also possible for an intermediate adapter to be arranged between endoscope and camera head. The known arrangements make use of a transparent window or a transparent protective film to separate the sterile side and the non-sterile side.

Sterile adapters with a transparent window are known from U.S. Pat. No. 5,498,230 A and from EP 0 955 014 A1.

U.S. Pat. No. 5,882,295 A and U.S. Pat. No. 5,433,221 A disclose transparent sterile covers which produce a sterile barrier between endoscope and camera head with a transparent film.

The object of the invention is to make available an improved sterile adapter.

For this purpose, according to the invention, a sterile adapter for arrangement between a sterilizable endoscope and a camera head is proposed, comprising a distal end which is attachable to the endoscope, and a proximal end which is attachable to the camera head. The sterile adapter has a tube with a longitudinal axis, wherein a longitudinal portion of the tube has an internal diameter which is reduced in a cross-sectional plane perpendicular to the longitudinal axis and which forms a central opening which is dimensioned such that, while a free propagation of light is possible between the endoscope and the camera head along the longitudinal axis, it is nonetheless made difficult to reach through an interior of the sterile adapter enclosed by the tube, wherein the central opening of the longitudinal portion has an internal diameter of at most 10 mm, and wherein the tube has an internal diameter of less than 30 mm at its distal end, and a distance of the opening from the distal end of the tube is greater than half an internal diameter of the tube at the distal end thereof.

An advantage of the invention is that the sterile adapter permits a free propagation of light between endoscope and camera head, such that an endoscope image quality captured by the camera head is not reduced by the adapter. In particular, the free propagation of light through the opening of the sterile adapter ensures that no soiling, streaking or birefringence at optical components of the sterile adapter adversely affect the image quality, as may happen, for example, in the case of transparent windows or transparent protective films known from the prior art. Thus, free propagation of light signifies hereinbelow that light does not have to refract at solid materials such as a film or a window, and instead at least parts of a light beam propagating between camera head and endoscope are unobstructed.

Since the sterile adapter according to the invention has no surfaces through which light is guided, there is no need for cleaning such optical surfaces in the context of preparation for use of the sterile adapter.

In addition, transmission of microorganisms from the non-sterile camera head is avoided, since contact with the camera head through the sterile adapter is made difficult. Particularly in a sterile environment, for example in the context of a medical operation, accidental contact with the non-sterile camera head is avoided. This can also be advantageous for changing the endoscope during an operation, without exposing a sterile operating environment to the microorganisms of the camera head.

The sterile adapter thus permits free propagation of light in combination with protection against a sterile environment coming into contact with the non-sterile camera head.

The diameter of the opening is advantageously chosen so as to avoid inadvertently reaching through the opening to the non-sterile camera head.

Since the distance of the opening from the distal end of the tube is greater than half an internal diameter of the tube at the distal end thereof, an opening angle of the sterile adapter is described which is present between the distal end and the central opening, which angle measures at most 90° starting from a center point of the opening.

Particularly for non-circular cross sections, the term diameter or internal diameter signifies hereinbelow a minimum extent in a direction perpendicular to the longitudinal axis. However, the terms diameter and internal diameter are not intended to imply that the opening or the tube are circular. While the opening is circular in a preferred embodiment, the opening in other embodiments is polygonal or round. In particular, the opening can also be rectangular, ellipsoid or star-shaped.

Preferred embodiment variants of the sterile adapter are proposed below.

In a particularly preferred embodiment variant, the central opening of the longitudinal portion has an internal diameter of between 1 mm and 10 mm, in particular of between 4 mm and 8 mm. Since typical beam widths of the endoscope are in the region of one millimeter, this embodiment variant is particularly advantageous both for permitting a free propagation of light between endoscope and camera head and also for avoidance of reaching through, for example by the fingers of a person using the endoscope. A smaller internal diameter reduces the possibility of coming into contact with the camera head. However, for stereo-endoscopic applications, internal diameters of between 4 mm and 8 mm may be better suited than internal diameters of between 1 mm and 3 mm, since two beam paths have to be imaged by the camera head.

In the direction of the longitudinal axis, the tube preferably has an extent of at least 10 mm and in particular of more than 30 mm. This increases a lengthwise dimension of the adapter and therefore avoids the possibility of reaching through into the volume of the sterile adapter surrounded by the tube. An extent of the tube according to this embodiment. variant can have the effect that, despite inadvertent contact with the inner contour of the sterile adapter, no contact is made with the non-sterile camera head.

The longitudinal portion of reduced internal diameter preferably has a width of between 0.5 mm and 2 mm in the direction of the longitudinal axis.

In a particularly preferred embodiment variant, the longitudinal portion of reduced internal diameter is configured as a pinhole stop, i.e. the extent of the longitudinal portion of reduced internal diameter in the longitudinal direction is smaller than the diameter of the central opening. The sterile adapter of this embodiment can be produced particularly easily, for example by an extrusion process.

The sterile adapter according to the invention is preferably produced from stainless steel. In other variants, the sterile adapter is produced from aluminum, rigid plastic or another polymer.

For production of the sterile adapter, it is particularly advantageous if it has a monolithic structure. No additional securing means then has to be provided for securing the longitudinal portion or for securing a pinhole stop to the tube.

In a particularly preferred embodiment variant, the sterile adapter is configured to be largely rotationally symmetrical with respect to the longitudinal axis. This permits particularly simple production. In this embodiment variant, the tapering of the inner contour is preferably configured as a pinhole stop. Deviations from a rotationally symmetrical configuration can be provided, for example, by a connection for connecting to the sterilizable endoscope or to the camera head. A sterile adapter of this embodiment variant can, for example, have an outer jacket surface of the tube, which surface extends in the longitudinal direction parallel to the longitudinal axis or is conically shaped.

In one embodiment variant, the tube has, in the direction of the longitudinal axis, an extent that is 0.8 times the maximum internal diameter of the tube in a direction perpendicular to the longitudinal axis.

This ratio between the extent of the tube and the maximum internal diameter has the effect that, in this embodiment variant, the opening angle is less than 70°. This can avoid accidental reaching through to an interior of the sterile adapter enclosed by the tube.

In a particularly preferred embodiment variant, the opening is formed at the proximal end of the sterile adapter. While contact with the camera head is still avoided in this embodiment variant, a reduced internal diameter formed near the camera head at the proximal end of the sterile adapter ensures, by comparison with a tapering at the distal end of the sterile adapter, a greater aperture of the camera head attached to the sterile adapter. This can have an advantageous effect on the image quality of endoscope data captured by the camera head.

In a further embodiment variant, a movable shutter is arranged on the tube in the longitudinal portion and is designed to adopt an open shutter position when the sterile adapter is mechanically connected to the endoscope, and to adopt a closed shutter position when the sterile adapter is not mechanically connected to the endoscope. Here, the closed shutter position obstructs the free propagation of light between the distal end and the proximal end of the sterile adapter, while the open shutter position permits the free propagation of light. In this embodiment variant, the sterile environment is protected particularly effectively against microorganisms of the non-sterile camera head. Thus, when the endoscope is changed, the danger of contact with the camera head is reduced both by the tube and the reduced internal diameter in the region of the longitudinal portion and also by the movable shutter. The movable shutter can largely exclude accidental contact with the non-sterile camera head, since contact of this camera head is not usually possible when an endoscope is attached and the shutter thus open. In addition, the sterile adapter of this embodiment variant has the advantage of unobstructed propagation of light between endoscope and camera head.

In one embodiment variant, the movable shutter is mechanically realized by a spring which is connected to a flap mounted on an inner contour of the longitudinal portion, in such a way that, when the endoscope is applied, a spring force of the spring folds open the flap, and wherein the spring force of the sprint is reduced when the endoscope is released from the sterile adapter, which causes the flap to fold shut. In a further embodiment variant, the movable shutter is formed by at least one plane diaphragm element, wherein the application of the endoscope to the sterile adapter causes a displacement of the aperture element in a plane perpendicular to the longitudinal axis, as a result of which a free propagation of light between endoscope and camera head is permitted. By contrast, in this embodiment variant, the release of the endoscope ensures a displacement of the diaphragm element in the plane perpendicular to the longitudinal axis, such that the shutter is transferred to the closed shutter position. The diaphragm element is preferably designed in this case as a leaf diaphragm with a number of further diaphragm elements on the sterile adapter.

In a preferred embodiment variant, the sterile adapter and the endoscope and/or the camera head are attachable to each other via a plug connection formed in each case by the tube. This plug connection can have an additional securing means, for example a releasable latching mechanism, in order to prevent inadvertent release of the endoscope or of the camera head from the sterile adapter.

In a further embodiment variant, the sterile adapter and the endoscope and/or the camera head are attachable to each other via a screw connection formed in each case by the tube. A screw connection advantageously permits a secure connection between sterile adapter and/or endoscope, wherein a risk of inadvertent release of the endoscope or of the camera head is particularly low.

In a further embodiment variant, the sterile adapter is secured to the camera head via a screw connection and to the endoscope via a plug connection. This can allow the endoscope to be changed easily and quickly in a sterile environment via the plug connection. Moreover, the sterile adapter is screwed onto the camera head, such that it is possible to avoid inadvertent release of the camera head from the sterile adapter and the resulting contamination of a sterile environment with the camera head.

The invention will now be explained in more detail on the basis of illustrative embodiments and with reference to the figures, in which:

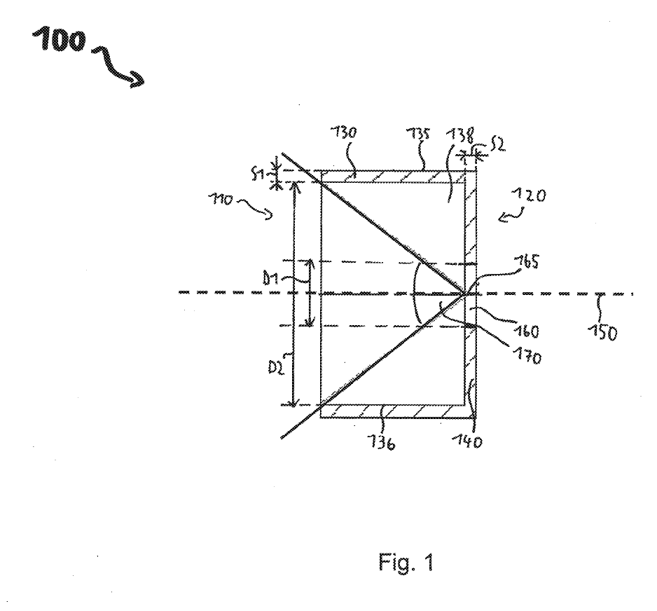

FIG. 1 shows a cross section through an illustrative embodiment of the sterile adapter according to the invention;

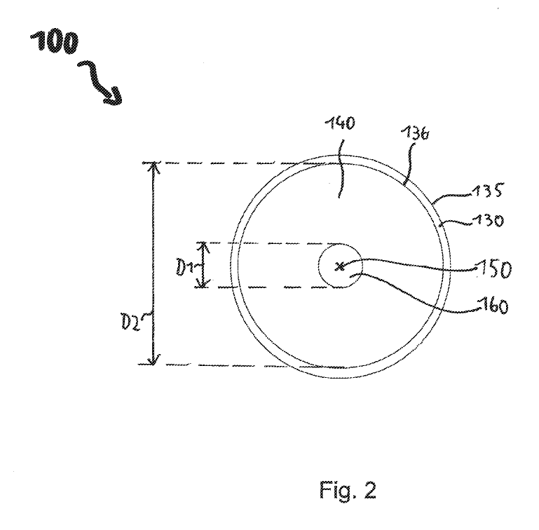

FIG. 2 shows a front view of the illustrative embodiment of the sterile adapter according to the invention from FIG. 1;

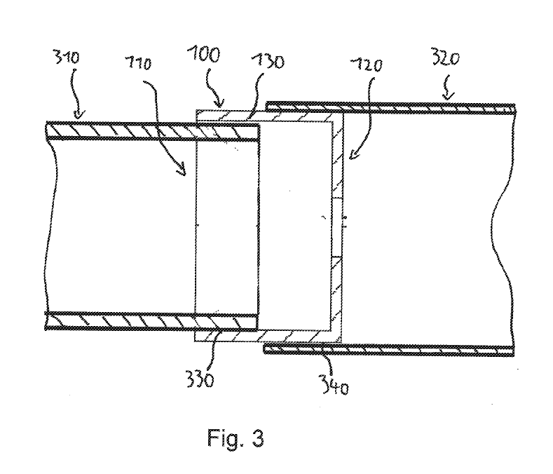

FIG. 3 shows a schematic view of the cross section from FIG. 1 with attached endoscope and camera head.

FIG. 1 shows a cross section through an illustrative embodiment of the sterile adapter 100 according to the invention.

The sterile adapter 100 has a distal end 110, to which an endoscope can be attached, and a proximal end 120, to which a camera head can be attached. A tube 130 with a longitudinal axis 150 forms a jacket surface 135 of the sterile adapter 100 with an inner contour 136. A longitudinal portion 140 has an internal diameter D1 which is reduced in a cross-sectional plane perpendicular to the longitudinal axis 150 and which forms a central opening 160. The central opening 160 is dimensioned such that, while free propagation of light is permitted between the endoscope at the distal end 110 and the camera head at the proximal end 120 along the longitudinal axis 150, it is nonetheless made difficult to reach through an interior 138 of the sterile adapter 100 enclosed by the tube 130. The central opening 160 of the longitudinal portion 140 has an internal diameter D1 of at most 10 mm, wherein the tube 130 has an internal diameter D2 of less than 30 mm at its distal end 110, and a distance of the opening 160 from the distal end 110 of the tube 130 is greater than half an internal diameter D2 of the tube 130 at the distal end 110 thereof.

Through its extent as far as the distal end 110, starting from a center point 165 of the opening 160, the tube 130 describes an opening angle 170 of the sterile adapter 100. This opening angle 170 is configured such that it measures at most 90°. The center point 165 here is the geometric centroid of a form, described by the opening 160, of a distal side of the longitudinal portion 140 directed toward the distal end 110 of the sterile adapter 100.

In the illustrative embodiment shown, the internal diameter D1 is between 4 mm and 8 mm. Furthermore, the extent of the tube 130 is at least 10 mm, preferably 18 mm.

The jacket surface 135 formed by the tube 130 is axially symmetrical with respect to the longitudinal axis 150 and surrounds the longitudinal axis in the shape of a cylindrical jacket, although it can also, for example, be completely or partially in the shape of a cylindrical jacket or also completely or partially conical. Outside a region of its tapering caused by the reduced internal diameter D1, the inner contour 136 is likewise parallel to the longitudinal axis 150. A wall thickness S1 of the tube 130 between jacket surface 135 and inner contour 136 is between 0.5 mm and 2 mm.

The longitudinal portion 140 is formed, by means of the reduced internal diameter D1, as a pinhole stop with the opening 160. The pinhole stop is formed perpendicularly with respect to the longitudinal axis 150. That part of the inner contour 140 lying inside the opening 160 is parallel to the jacket surface 135. The pinhole stop formed by the region of reduced internal diameter D1 has a thickness S2 of between 0.5 mm and 2 mm, wherein the thickness S2 in the illustrative embodiment shown is identical to the thickness S1. The longitudinal portion 140 is formed here at the proximal end 120 of the sterile adapter 100 in such a way that the sterile adapter 100 has a monolithic form and is rotationally symmetrical with respect to the longitudinal axis 150, although this is only apparent from FIG. 2.

In the illustrative embodiment shown, the pinhole stop formed by the longitudinal portion 140 is configured at a proximal edge of the proximal end 120 flush with the circumferential jacket surface 135. In an illustrative embodiment not shown, the longitudinal portion 140 of reduced internal diameter D1 has an identical spacing between the proximal end and the distal end of the sterile adapter.

FIG. 2 shows a front view of the illustrative embodiment of the sterile adapter 100 according to the invention from FIG. 1.

In contrast to the cross section in FIG. 1, the sterile adapter 100 is shown in FIG. 2 as being rotationally symmetrical with respect to the longitudinal axis 150. Here, the opening 160 is shaped as a circle and has the internal diameter D1. In other illustrative embodiments not shown, the opening has a polygonal or ellipsoid shape, in particular a rectangular shape.

In view of the rotational symmetry, the tube 130 has a circular shape. It is delimited by the circularly shaped jacket surface 135 and the likewise circular inner contour 136, which has the internal diameter D2 at the distal end.

The longitudinal portion 140 of reduced internal diameter D1 is formed as a pinhole stop with the opening 160.

FIG. 3 shows a schematic view of the cross section of the sterile adapter 100 from FIG. 1 with an attached sterilizable endoscope 130 and camera head 320.

The endoscope 310 is plugged into the sterile adapter 100 via a first plug connection 330 at the distal end 110. In the plugged-in state shown, the tube 130 surrounds a part of the endoscope 310.

The camera head 320 is plugged onto the sterile adapter 100 via a second plug connection 340 at the proximal end 120 of the sterile adapter 100, such that a part of the camera head 320 surrounds the tube 130. In the illustrated example of the operation of the sterile adapter 100 with attached endoscope 310 and camera head 320, a region of the camera head 320 accessible from the outside and surrounding the sterile adapter 100 is sterile, while an inner region of the camera head 320 protected by the sterile adapter is non-sterile.

In an illustrative embodiment not shown, a movable shutter is arranged on the tube in the region of the longitudinal portion and is designed to adopt an open shutter position when the sterile adapter is attached to the endoscope, and to adopt a closed shutter position when the sterile adapter is not attached to the endoscope. Here, the closed shutter position obstructs a free propagation of light between the endoscope and the camera head, and the open shutter position permits the free propagation of light.

In a further illustrative embodiment not shown, the first and/or the second plug connection is configured such that a part of the endoscope and/or a part of the camera head surrounds the tube in the attached state, wherein furthermore a web on the jacket surface of the tube is formed in a plane perpendicular to the longitudinal axis, such that the web obstructs a movement of the endoscope and/or of the camera head along the tube in the direction of the longitudinal axis and thus limits a respective depth of connection of the first and/or second plug connection. The web can in particular prevent the endoscope and camera head from touching each other on the jacket surface of the sterile adapter in the attached state.

### LIST OF REFERENCE SIGNS

100 sterile adapter

110 distal end

120 proximal end

130 tube

135 jacket surface

136 inner contour

138 enclosed interior

140 longitudinal portion

150 longitudinal axis

160 opening

165 center point

170 opening angle

310 sterilizable endoscope

320 camera head

330 first plug connection

340 second plug connection

D1 internal diameter of the opening

D2 diameter of the inner contour

S1 thickness of the tube

S2 thickness of the pinhole stop

1 A sterile adapter for arrangement between a sterilizable endoscope and a camera head, comprising a distal end which is attachable to the endoscope, and a proximal end which is attachable to the camera head, wherein the sterile adapter has a tube with a longitudinal axis, wherein a longitudinal portion of the tube has an internal diameter which is reduced in a cross-sectional plane perpendicular to the longitudinal axis and which forms a central opening which is dimensioned such that, while a free propagation of light is possible between the endoscope and the camera head along the longitudinal axis, it is nonetheless made difficult to reach through an interior of the sterile adapter enclosed by the tube, wherein the central opening of the longitudinal portion has an internal diameter of at most 10 mm, and wherein the tube has an internal diameter of less than 30 mm at its distal end, and a distance of the central opening from the distal end of the tube is greater than half an internal diameter of the tube at the distal end thereof.

2. The sterile adapter as claimed in claim 1, in which the central opening of the longitudinal portion has an internal diameter of between 1 mm and 10 mm, in particular of between 4 mm and 8 mm.

3. The sterile adapter as claimed in claim 1, in which the tube has, in the direction of the longitudinal axis, an extent of at least 10 mm and preferably of more than 30 mm.

4. The sterile adapter as claimed in claim 1, in which the longitudinal portion of reduced internal diameter has a width of between 0.5 mm and 2 mm in the direction of the longitudinal axis.

5. The sterile adapter as claimed in claim 1, in which the longitudinal portion of reduced internal diameter is configured as a pinhole stop.

6. The sterile adapter as claimed in claim 1, which sterile adapter is largely rotationally symmetrical with respect to the longitudinal axis.

7. The sterile adapter as claimed in claim 1, in which the sterile adapter is of monolithic form.

8. The sterile adapter as claimed in claim 1, in which the tube has, in the direction of the longitudinal axis, an extent that is greater than 0.8 times the maximum internal diameter of the tube in a direction perpendicular to the longitudinal axis.

9. The sterile adapter as claimed in claim 1, in which the opening is formed at the proximal end of the sterile adapter.

10. The sterile adapter as claimed in claim 1, in which a movable shutter of the opening is arranged on the tube in the longitudinal portion and is designed to adopt an open shutter position when the sterile adapter is mechanically connected to the endoscope, and to adopt a closed shutter position when the sterile adapter is not mechanically connected to the endoscope, wherein the closed shutter position obstructs the free propagation of light between the endoscope and the camera head, and the open shutter position permits the free propagation of light.

11. The sterile adapter as claimed in claim 1, wherein the sterile adapter and the endoscope and/or the camera head are attachable to each other via a plug connection formed in each case by the tube.

12. The sterile adapter as claimed in claim 1, wherein the sterile adapter and the endoscope and/or the camera head are attachable to each other via a screw connection formed in each case by the tube.
